(19) United States
(12) Patent Application Publication
(10) Pub. No.: US 2026/0193166 A1
(43) Pub. Date: Jul. 9, 2026

(54) HYDROGENATION PROCESS AND REACTION SYSTEM FOR A HYDROGENATION PROCESS

(71) Applicants: BASF SE, Ludwigshafen (DE); SHANGHAI BASF POLYURETHANE COMPANY LTD., Shanghai (CN)

(72) Inventors: Zheng Yi YU, Shanghai (CN); Zhi Jiang GOU, Shanghai (CN); Man Gyun PARK, Yeosu (KR); Stefan ENGELS, Ludwigshafen am Rhein (DE); Matthias HINRICHS, Ludwigshafen am Rhein (DE); Steffen OEHLENSCHLAEGER, Ludwigshafen am Rhein (DE); Oliver BEY, Ludwigshafen am Rhein (DE); Hans SCHUYTEN, Antwerp (BE)

(21) Appl. No.: 19/132,016

(22) PCT Filed: Nov. 23, 2023
(86) PCT No.: PCT/EP2023/082885

§ 371 Date: May 22, 2025

(30) Foreign Application Priority Data
Nov. 24, 2022 (WO) PCT/CN2022/134049

(51) Int. Cl. C07C 209/36; B01J 8/18; B01J 8/22

(52) U.S. Cl. CPC C07C 209/36; B01J 8/1827; B01J 8/1836; B01J 8/1881; B01J 8/228; B01J 2208/00115; B01J 2208/00176; B01J 2208/00256; B01J 2208/00902

(57) ABSTRACT

In a first aspect, the invention relates to a hydrogenation process for the hydrogenation of a compound comprising reacting the compound in a liquid medium with hydrogen in the presence of a heterogeneous hydrogenation catalyst in a reaction vessel, the process comprising: (i) transferring at least a part of the thermal energy generated in the reaction vessel to a cooling medium, preferably a cooling medium stream CMS1, obtaining a cooling medium stream CMS2, which has an increased thermal energy content compared to CMS1; and (ii) transferring at least a part of the thermal energy comprised in cooling medium stream CMS2 to a heat transfer medium stream HTMS1 in a heat pump HP, thereby obtaining a heat transfer medium stream HTMS2, which has an increased thermal energy content compared to HTMS1.

A second aspect of the invention is directed to a reaction system for hydrogenation of a compound, preferably for converting a compound having at least one nitro group into the corresponding compound having at least one amino group by hydrogenation.

In a first aspect, the invention relates to a hydrogenation process for the hydrogenation of a compound, comprising reacting the compound in a liquid medium with hydrogen in the presence of a heterogeneous hydrogenation catalyst in a reaction vessel, the process comprisesing: (i) transferring at least a part of the thermal energy generated in the reaction vessel to a cooling medium, preferably a cooling medium stream CMS1, obtaining a cooling medium stream CMS2, which has an increased thermal energy content compared to CMS1; and (ii) transferring at least a part of the thermal energy comprised in cooling medium stream CMS2 to a heat transfer medium stream HTMS1 in a heat pump HP, thereby obtaining a heat transfer medium stream HTMS2, which has an increased thermal energy content compared to HTMS1. A second aspect of the invention is directed to a reaction system for hydrogenation of a compound, preferably for converting a compound having at least one nitro group into the corresponding compound having at least one amino group by hydrogenation.

Hydrogenation is a type of reaction that is used in many fields of applications. The reaction is characterized by the use of hydrogen, which is used as a reducing agent for a substrate, typically with additional use of a catalyst. Common reactants used in a hydrogenation reaction are, for example, alkenes, alkynes, aldehydes, ketones, esters, carboxylic acids and compounds having nitro groups. From the later, dinitro toluene is often used for hydrogenation reactions since the respective reaction product toluene diamine is the precursor of toluene diisocyanate, which is in turn the relevant isocyanate monomer in the preparation of polyurethane.

In existing technology, toluene diamine is produced by hydrogenation of dinitro toluene, wherein the reaction is strongly exothermic and sets a considerable amount of thermal energy free. Said reaction heat has to be removed, otherwise the reaction could not be carried out in a controlled manner for a prolonged period of time. Common means for removing the reaction heat are cooling systems, which normally use water as cooling medium. In one or more cooling loops, the reaction heat is taken off and for example then the cooling medium is disposed, since the cooling water in the end is discharged into waters. Especially nowadays, there is an overall demand for energetically efficient systems, i.e. systems, which are capable of not wasting but rather using generated energy for a meaningful purpose. Since steam, especially water steam, is an efficient energy carrier, which is used in many industrial processes, it could be considered to transfer the reaction heat of a hydrogenation reaction to steam.

Reactor setups for hydrogenation of dinitro toluene are described in the art, for example, WO 00/30743 A1 discloses a reactor of cylindrical construction for the continuous performance of solid gas-liquid, liquid-liquid or gas-liquid reactions, having a downwardly directed jet nozzle arranged in the upper reactor region, via which the starting materials and the reaction mixture are fed, and having a draw-off preferably in the lower reactor region, via which the jet nozzle is fed. Further described is a continuous process for carrying out gasliquid or gas liquid-solid reactions in said reactor.

WO 00/35852 A1 is related to a process for preparing amines by hydrogenation of nitro compounds, characterized in that the hydrogenation is carried out in a vertical reactor whose length is greater than diameter with a downwardly directed jet nozzle arranged in the upper region of the reactor, via which the feeding materials and the reaction mixture are fed, and with a draw-off at any point in the reactor, via which the reaction mixture is fed in an external circuit back to the reactor.

However, the reaction temperature in most of the existing reactor setups is not high enough to be used efficiently, for example, for generation of steam with a higher grade, which can be used economically.

Thus, the problem underlying the present invention was to provide a way for overcoming the lack of current technology, especially to provide a process and a reaction system for recovering reaction heat from hydrogenation more efficiently, for example, by providing steam with a sufficiently high grade.

### 1^{st} Aspect-Hydrogenation Process

In a first aspect, the invention is directed to a hydrogenation process for the hydrogenation of a compound comprising reacting the compound in a liquid medium with hydrogen in the presence of a heterogeneous hydrogenation catalyst in a reaction vessel, the process comprising
(i) transferring at least a part of the thermal energy generated in the reaction vessel to a cooling medium, preferably a cooling medium stream CMS1, obtaining a cooling medium stream CMS2, which has an increased thermal energy content compared to CMS1;
(ii) transferring at least a part of the thermal energy comprised in cooling medium stream CMS2 to a heat transfer medium stream HTMS1 in a heat pump HP, thereby obtaining a heat transfer medium stream HTMS2, which has an increased thermal energy content compared to HTMS1.

In some preferred embodiments, the hydrogenation process is a process for the hydrogenation of a compound having at least one nitro group into the corresponding compound having at least one amino group comprising reacting the compound having at least one nitro group in a liquid medium with hydrogen in the presence of a heterogeneous hydrogenation catalyst in a reaction vessel.

As indicated in further detail below, the hydrogenation process allows a favorable energetic balance in that at least 20% of the thermal energy generated in the reaction vessel are transferred, via the cooling medium and the heat pump, to the heat transfer medium. In embodiments where the heat pump is an absorption heat pump, preferably an absorption heat pump of category II, in the range of from 20 to 50% of the thermal energy generated in the reaction vessel are transferred, via the cooling medium and the heat pump, to the heat transfer medium. In alternative embodiments where the heat pump is a compression heat pump, preferably a compression heat pump to generate steam (steam generator), in the range of from 20 to 100%, preferably in the range of from 70 to 100%, of the thermal energy generated in the reaction vessel are transferred, via the cooling medium and the heat pump, to the heat transfer medium.

### Heat Pump

In some preferred embodiments of the hydrogenation process, the heat pump of (ii) is an absorption heat pump, preferably an absorption heat pump of category II, or a compression heat pump, more preferably the heat pump of (ii) is an absorption heat pump of category II.

### Absorption Heat Pump

Absorption heat pumps offer an opportunity to upgrade a low or medium temperature heat source to a useful temperature level. Therefore absorption heat pumps use low or medium temperature heat sources as driving heat source and use the effect of absorption heat to increase the/a temperature level. In the field of absorption heat pumps, there are Type I heat pumps and Type II heat pumps, the later also being called an absorption heat transformer. In heat pumps of type I the condenser temperature is higher than evaporator temperature. The absorption heat pump extracts the heat of waste heat and outputs a mediumtemperature heating media (preferably water) that is higher than the waste heat. Typical is a medium heating media that is in the range of from 30 to 60° C. higher than the heat waste temperature or and provides a heating media with a temperature in the range of from 60 to 95° C. In heat pumps of type II the condenser temperature is lower than evaporator temperature. The type II absorption heat pump uses the heat of the medium-temperature waste heat intelligently, output high-temperature heat medium (preferable water steam) in the range of from 25 to 50° C. higher than medium temperature waste heat. Absorption heat pumps are transforming a heating media (preferable water) to steam and provide steam with temperature above 100° C. Thereby absorption heat pumps of type II upgrade a portion of the heat from the heat source to a higher temperature level as useful heat, while rejecting the rest of the heat at low temperature to the heat sink (e.g. ambient air, cooling water). Upgrading up to 50% of the available waste heat is possible. An absorption heat pump comprises one or more stage(s), wherein more than one stage is used in order to realize a wider temperature range. In order to obtain greater temperature lifts (>50 K), a heat pump with higher number of stages must be used. With increasing temperature lift, the quantity of energy which may be upgraded decreases. For example, for much higher temperature lifts, the triple-stage water/LiBr absorption heat pump may be used to attain temperature lifts of up to 145 K by upgrading about 20% of the available waste heat.

An absorption heat pump is based on the use of a pair of working fluids. One working fluid acts as a refrigerant, the other as a solvent. The refrigerant must always have the higher vapor pressure, since the solvent should remain in the liquid phase when the refrigerant is expelled in the generator. Possible working fluid pairs are: water/lithium bromide (LiBr) (water as refrigerant), ammonia/water (ammonia as refrigerant), ammonia/lithium nitrate (LiNO3) and ammonia/sodium thiocyanate, ammonia/ionic liquids, water/ionic liquids, methanol or ethanol/ionic liquid and trifluoroethanol/tetraethylene glycol dimethyl ether.

In the context of the present invention, it is preferred that the absorption heat pump of category II uses the working fluid pair of water and LiBr, wherein water acts as the refrigerant. The working principle of an absorption heat transformer is that the drive heat flow, in the present case the thermal energy coming from cooling medium stream CMS2 (or CMS4 or the combined stream of CSM2 and CSM5, see further below for details), is absorbed in the generator and evaporator, whereafter cooling medium stream CMS3, which has a decreased thermal energy content compared to CMS2, leaves the generator. The refrigerant is evaporated in the evaporator at elevated pressure and is then fed to the absorber, where it is absorbed by the solvent. The resulting mixture is expanded via a throttle and fed to the generator. There, the solvent is expelled at lower pressure by the driving heat. The solvent is brought back to increased pressure via a pump and fed to the absorber. In many cases, it is preheated via a heat exchanger by the heat of the mixture stream leaving the absorber. The expelled refrigerant is fed to the condenser. There it condenses at a significantly lower temperature due to the low pressure. The liquid refrigerant is then brought back to higher pressure by a second pump and fed to the evaporator. The usable heat supplied by the heat transformer is the heat of solution of the refrigerant in the solvent released in the absorber, which is transferred to heat transfer medium stream HTMS1, thereby obtaining heat transfer medium stream HTMS2, which has an increased thermal energy content compared to HTMS1.

### Compression Heat Pump

Compression heat pumps offer an opportunity to upgrade a low or medium temperature heat source to a useful temperature level. Therefore, compression heat pumps are using mechanical work as driving source to lift the temperature level. An (electrically driven) compression pump operates with a closed refrigerant circuit, wherein the refrigerant is selected from the group consisting of ammonia, water, chlorofluorocarbon, hydrochlorofluorocarbon, hydrofluorocarbon, hydrofluoroolefin, hydrochlorofluoroolefin, hydrocarbon, perfluoro (2-methyl-3-pentanone) and mixtures of two or more thereof. Also gaseous refrigerants are suitable, e.g. carbondioxide or other gases. Suitable refrigerants are known to the skilled person and are disclosed, for example, in C. Arpagaus et al. (C. Arpagaus et al., Energy 152 (2018), pages 985 to 1010). In case of liquid refrigerants the initially liquid refrigerant enters the evaporator. In the region of the evaporator, the pressure is kept low, typically at a pressure in the range of 0 to 30 bara by a compressor so that the refrigerant boils and consequently evaporates. During this process, the refrigerant cools down because of the required heat of evaporation. Since the evaporator is designed as a heat exchanger, i.e., it allows heat exchange of the refrigerant with an external medium, here the warm cooling medium stream CMS2 (or CMS4 or the combined stream of CSM2 and CSM5, see further below for details), thermal energy (heat) now flows from the cooling medium stream CMS2 into the refrigerant, which significantly reduces its cooling and facilitates further evaporation. Cooling medium stream CMS3, which has a decreased thermal energy content compared to CMS2, thereafter leaves the region of the evaporator. The evaporated refrigerant is initially slightly cooler (in the range of from 2 to 40 K, preferably in the range of from 5 to 20 K, cooler) than the cooling medium stream CMS2. It then passes through the compressor (e.g. a screw, scroll, piston or turbo compressor), which brings it to a much higher pressure, typically a pressure in the range of 1 to 100 bara, which also causes a temperature increase in the refrigerant. The refrigerant is then forced to condense again in a second heat exchanger, the condenser. There, the refrigerant releases thermal energy (mainly condensation heat) at a higher temperature, which is transferred to heat transfer medium stream HTMS1 hereby obtaining heat transfer medium stream HTMS2, which has an increased thermal energy content compared to HTMS1. Condensation despite the higher temperature in the condenser is possible because of the pressure increase by the compressor. The condensed refrigerant is depressurized by an expansion valve and send as a liquid refrigerant back to the evaporator. In case of gaseous refrigerants, the same principles apply but without changes of the aggregate state only with increase or decrease of temperature of the gaseous refrigerant. In case of gaseous the gas cooled down refrigerant is depressurized by an expansion valve or expansion turbine and send as a partly or fully overcritical media (e.g. gases like carbondioxide) back to the evaporator.

For sake of completeness, it has to be noted that herein, pressures indicated in “barg” are related to gauge pressure. Since gauge pressure is measured against the ambient pressure, each pressure value (or range) indicated in barg it is equal to absolute pressure minus atmospheric pressure. “bara” means the absolute pressure in bar.

A heat pump is characterized by its coefficient of performance (COP) and its quality grade eta.

COP is calculated based on the equitation (I)

$$COP = Q / W \qquad ( I )$$

wherein “Q” is the heat transferred into the heat transfer media (usable heat: HTMS2-HTMS1) and “W” is the work of the compressor (work spent, elelectric energy) or driving heat.

The quality grade eta is calculated based on equitation (II)

$$eta = {COP}_{HP} / {COP}_{Carnot} , \qquad ( II )$$

wherein COP_{HP} is the coefficient of performance of the heat pump and COP_{Carnot}: is the ideal coefficient of performance calculated based on a Carnot process.

The process of the present invention enables a COP_{HP} in the range of from 1 to 8, preferably in the range of from 1.2 to 7.5, and a quality grade eta in the range of from 30 to 65% depending on the used compressor type (Piston-, Screw-, Turbocompressor).

### Heat Transfer Medium

In some embodiments of the hydrogenation process, the heat transfer medium comprises water, preferably at least 90 weight-%, more preferably at least 95 weight-%, more preferably at least 99 weight-% of the heat transfer medium are water, each based on the total weight of the heat transfer medium being 100 weight-%. In some embodiments of the hydrogenation process, at least 90 weight-% of heat transfer medium stream HTMS1 are in liquid state and at least at least 90 weight-% of heat transfer medium stream HTMS2 are in gaseous state (H₂O_{gaseous}, water steam). Heat transfer medium stream HTMS1, which enters the heat pump HP, has preferably a temperature T1 in the range of from >0 to 140° C., more preferably in the range of from 20 to 140° C., more preferably in the range of from 90 to 120° C. HTMS1 has a pressure p₁, which is >p₂, wherein the value of p₁ and the absolute value of the difference between p₂ and p₁ depends on the reaction apparatus, the setup and arrangement of the lines etc., which a person skilled in the art is familiar with.

In some embodiments of the hydrogenation process, the heat transfer medium stream HTMS2 has a pressure p₂ in the range of from 0 to 20 barg, preferably in the range of from 0.1 barg to 10 barg. Heat transfer medium stream HTMS2 preferably has a temperature T₂, which is equal or greater than the temperature at the boiling point (TBP) of the heat transfer medium at a pressure p₂.

In some embodiments of the hydrogenation process, the heat transfer medium stream HTMS2 having a pressure p₂ is compressed, preferably by mechanical compression, to give a heat transfer medium stream HTMS2-1 having an increased pressure p₂-1 compared to p₂, wherein p₂-1 is preferably a pressure in the range of from 3 to 40 barg. Heat transfer medium stream HTMS2-1 preferably has a temperature T₂₋₁, which is >=the temperature at the boiling point (TBP) of the heat transfer medium at a pressure p₂-1+3 K.

Especially if the heat transfer medium stream HTMS2 has a low pressure, for example, is water steam stream having a pressure p₂ in the range of from 0 to 8 barg, it is advantageous to do a compression by an additional mechanical steam compressor to pressurize the steam to pressures p₂-1 in the range of from p₁₊₄ barg to 40 barg. Even if additional electrical power input is needed for the additional mechanical compression, the overall energetic balance is still favorable.]

In some preferred embodiments, HTMS2 and/or HTMS2-1, each partially or totally, is/are used for heating in the process where the reaction takes place, including reaching and/or maintaining the temperature necessary for carrying out the reaction in the reaction vessel, and/or for heating in a further process, which is preferably a process prior to or subsequent to the preparation of the compound having at least one amino group, more preferably a subsequent process, wherein the compound having at least one amino group is converted into a compound having at least one isocyanate group or a prior process, in which the compound comprising at least one nitro group is generated. Further the generated steam can be used in a steam net at other production plants for heating or can be sold to a third party.

### Cooling Medium

In some embodiments of the hydrogenation process, the cooling medium is selected from the group consisting of water, air, solvent and mixtures of two or more thereof, wherein the cooling medium preferably comprises water, preferably at least 90 weight-%, more preferably at least 95 weight-%, more preferably at least 99 weight-% of the heat transfer medium are water, each based on the total weight of the cooling medium being 100 weight-%. A solvent in the context of the cooling medium means any solvent having the same boiling conditions, especially the same boiling point as the solvent(s) used in the reaction mixture, wherein the “same” includes a deviation by +5%. For example, if a C1 to C6 monoalcohol is used as solvent in the reaction mixture, especially ethanol and/or propanol, preferably iso-propanol, then a solvent having the same boiling conditions means a solvent having at 1013 mbar a boiling point in the range of from 78 to 86° C.

### Heat Exchanger—(Primary) Cooling System

In some embodiments of the hydrogenation process, transferring at least a part of the thermal energy generated in the reaction vessel to cooling medium stream CMS1 in (i) is done by use of a cooling system, which comprises a heat exchanger arranged within the reaction vessel or surrounding the reaction vessel at least partially and a circulating line for introducing CMS1 into the heat exchanger and for removing CMS2 from the heat exchanger. A heat exchanger is a device that transfers thermal energy from one material stream to another. While direct exchangers are known to the skilled person, the heat exchangers in the context of the present invention are preferably heat exchangers, wherein the material streams are not in direct contact with each other but are rather spatially separated, so that there is only an exchange of thermal energy possible. Heat exchangers are generally known, wherein suitable heat exchangers for being arranged within the reaction vessel can be, for example, tubes through which a cooling medium stream flows, the direction of which is preferably parallel to the reactor wall, plate heat exchangers which preferably run parallel to the reactor wall, or also double-lined tubes “tube in tube” closed at the bottom, so-called field tubes, can be used. For any heat exchanger which comprises at least two compartments, which are spatially separated but are in thermal contact, it is interchangeably which material stream flows through or is in which compartment.

In some embodiments of the hydrogenation process, transferring at least a part of the thermal energy comprised in cooling medium stream CMS2 to a heat transfer medium stream HTMS1 in a heat pump HP in (ii) is done so that a cooling medium stream CMS3 is obtained in (ii), which has the same or higher thermal energy content as CMS1, wherein preferably, CMS3 is reintroduced as CMS1 or at least as part thereof into the cooling system and the heat exchanger respectively.

In some embodiments of the hydrogenation process, the heat exchanger is selected from the group consisting of shell and tube heat exchanger, plate heat exchanger, field tube heat exchanger and coil heat exchanger.

Reaction vessel

In some embodiments of the hydrogenation process, the reaction vessel is a reactor selected from the group consisting of (multi) tubular reactor, stirred tank reactor and loop reactor, wherein the reaction vessel is preferably a loop reactor. Regarding combinations of heat exchangers and reaction vessels, a person skilled in the art is aware that specific heat exchangers are mainly suitable for specific reactor types. For example, for a loop reactor, a heat exchanger located in the reaction vessel such as a shell and tube heat exchanger is used in some preferred embodiments.

The reaction vessel is in some preferred embodiments a loop reactor. The loop reactor is a preferably vertical, preferably cylindrical, reactor. The loop reactor has a downward-facing jet nozzle arranged in the upper region of the reactor through which the starting materials and the reaction mixture are fed in, and has an outlet at any desired point of the reactor, preferably in the lower region, through which the reaction mixture is fed back to the jet nozzle in an external circuit by means of a conveying means, preferably a pump, and has flow reversal in the lower region of the reactor. The flow reversal and thus the formation of internal loop flow can be effected, in the case of take-off of the reaction mixture in the upper region of the reactor, by impact of the injected reaction mixture on the reactor base. In the case of the preferred take-off of the reaction mixture in the lower region of the reactor, the flow reversal is achieved by internals, in particular a baffle plate perpendicular to the reactor wall. The reactor contains one or more mixing chamber(s), which is/are preferably formed by cylindrical insertion tube(s) and is/are arranged within the reactor parallel to the reactor wall. The jet nozzle can be designed as a one- or two-component nozzle. In the case of the one-component nozzle, only the liquid reaction mixture is injected through the nozzle, and the (gaseous) hydrogen is fed into the reactor at any other desired point, but preferably at a point within the reaction mixture Suitable designs of a loop reactor are known and are, for example, described in WO 00/30743 A1, WO 00/35852 A1 or WO 2014/108352 A1. In some preferred embodiments, the loop reactor comprises a heat exchanger, preferably a heat exchanger located within the reactor. More preferably, said heat exchanger, equipped with suitable lines for introducing and removing a cooling medium stream, comprises one or more, preferably double-lined, tube(s) (field tubes), which are preferably arranged in the reactor substantially parallel to the reactor wall. “Substantially parallel” means that the tube(s) are arranged under an angle of +30°, preferably +20°, more preferably +10° with respect to the reactor wall. The cooling medium streams flows within the field tubes, which are surrounded on their outside by the reaction mixture in the reactor; a suitable setup is shown in FIG. 1 of WO 00/35852 A1. In some alternative embodiments, the reaction is conducted in tubes, whereas the cooling medium flow surrounds the tubes; a suitable setup is described shown in WO 2014/108352 A1.

The product, i.e. the compound having at least one amino group, is discharged from the system continuously or discontinuously, preferably continuously, at any desired point, but preferably at a point in the lower region of the reactor at its base or in particular from the external loop flow via a catalyst separation unit or without one. This separation unit can be a gravity separator, for example a settler, a suitable filter, for example a cross-flow filter, or a centrifuge. The catalyst can be separated from the product and then the catalyst can be fed back into the reactor system or discharged from the reactor system. The product is preferably discharged with retention of the catalyst. The product can then be purified by conventional and known methods, for example by distillation or extraction.

Loop reactor+Increase heat pump efficiency by increased temperatures (serial setup)

In some embodiments of the hydrogenation process, if the reaction vessel is a loop reactor, the process comprises:
(i.1) transferring at least a part of the thermal energy generated in the reaction vessel to a cooling medium, preferably a cooling medium stream CMS1, obtaining a cooling medium stream CMS2, which has an increased thermal energy content compared to CMS1;
(ii.a) feeding CSM2 to a heat exchanger installed in the circulation loop of the loop reactor, thereby transferring a further part of the thermal energy generated in the reaction vessel to CMS2 and obtaining a cooling medium stream CMS4, which has an increased thermal energy content compared to CMS2;
(ii.b) transferring at least a part of the thermal energy comprised in cooling medium stream CMS4 to a heat transfer medium stream HTMS1 in a heat pump HP, thereby obtaining a heat transfer medium stream HTMS2, which has an increased thermal energy content compared to HTMS1.

Preferably, the a cooling medium stream CMS3 is obtained in (ii), which has the same or higher thermal energy content as CMS1, wherein preferably, CMS3 is reintroduced as CMS1 or at least as part thereof into the cooling system and the heat exchanger respectively.

### Loop Reactor+Increase Heat Pump Efficiency by Increased Temperatures (Parallel Setup)

In some embodiments of the hydrogenation process, if the reaction vessel is a loop reactor, the process comprises:
(i) transferring at least a part of the thermal energy generated in the reaction vessel to a cooling medium, preferably a cooling medium stream CMS1, obtaining a cooling medium stream CMS2, which has an increased thermal energy content compared to CMS1;
(ii) sending a part of a cooling medium stream CMS3 (CMS3a) obtained from a heat pump PH to a heat exchanger installed in the circulation loop of the loop reactor, thereby transferring a part of the thermal energy generated in the reaction vessel to said part of cooling medium stream CMS3a and obtaining a partial cooling medium stream CMS5, which has an increased thermal energy content compared to CMS3;
(iii) combining partial cooling medium stream CMS5 with cooling medium stream CMS2 obtained in (i) thereby obtaining a combined cooling medium stream CMS2+CMS5;
(iv) feeding the combined cooling medium stream CMS2+CMS5 to the heat pump HP; and transferring at least a part of the thermal energy comprised in combined cooling medium stream CMS2+CMS5 to a heat transfer medium stream HTMS1 in a heat pump HP, thereby obtaining a heat transfer medium stream HTMS2, which has an increased thermal energy content compared to HTMS1.

It is understood that at the beginning of the process, after step (i) at least a part of the thermal energy comprised in cooling medium stream CMS2, i.e. without addition of CMS5, is transferred to a heat transfer medium stream HTMS1 in the heat pump HP, thereby obtaining a heat transfer medium stream HTMS2, which has an increased thermal energy content compared to HTMS1 and obtaining a cooling medium stream CMS3, which has a decreased thermal energy content compared to CMS2; later on, when the process has evolved, it is only the mixture of CMS2 and CMS5 that is fed to the heat pump HP.

In some embodiments of the hydrogenation process, another part of CMS3 (CMS3b) is reintroduced as CMS1 or at least as part thereof into the cooling system and the heat exchanger respectively.

### Temperatures

In some embodiments of the hydrogenation process, the cooling medium stream CMS2 or CMS4 or the combined stream of CSM2 and CSM5, which enters the heat pump HP has a temperature in the range of from 55 to 150° C. and the cooling medium stream CMS3, which leaves the heat pump HP has a temperature in the range of from 30 to 125° C.

### Energetic Balance

In some embodiments of the hydrogenation process, at least 20% of the thermal energy generated in the reaction vessel are transferred, via the cooling medium and the heat pump, to the heat transfer medium, preferably to heat transfer medium stream HTMS1 and heat transfer medium stream HTMS2 respectively.

In some embodiments of the hydrogenation process, where the heat pump is an absorption heat pump, preferably an absorption heat pump of category II, in the range of from 20 to 50% of the thermal energy generated in the reaction vessel are transferred, via the cooling medium and the heat pump, to the heat transfer medium, preferably to heat transfer medium stream HTMS1 and heat transfer medium stream HTMS2 respectively.

In some embodiments of the hydrogenation process, where the heat pump is a compression heat pump, preferably a compression heat pump to generate steam (steam generator), in the range of from 20 to 100%, preferably in the range of from 70 to 100% of the thermal energy generated in the reaction vessel are transferred, via the cooling medium and the heat pump, to the heat transfer medium, preferably to heat transfer medium stream HTMS1 and heat transfer medium stream HTMS2 respectively.

In some embodiments of the hydrogenation process, where the heat transfer medium stream HTMS2 is water steam (H₂O_{gaseous}), the ratio of the amount of steam generated in tons to the amount of thermal energy generated in the reaction vessel in MW (MWₕₑₐₜ) is in the range of from 0.3 to 7.0 tₛₜₑₐₘ/MWₕₑₐₜ.

In some embodiments of the hydrogenation process, where the heat pump is an absorption heat pump, preferably an absorption heat pump of category II, the ratio of the amount of steam generated in tons to the amount of thermal energy generated in the reaction vessel in MW is in the range of from 0.3 to 0.7 tₛₜₑₐₘ/MWₕₑₐₜ, preferably in the range of from 0.35 to 0.6 tₛₜₑₐₘ/MWₕₑₐₜ:

In some embodiments of the hydrogenation process, where the heat pump is a compression heat pump, preferably a compression heat pump to generate steam, the ratio of the amount of steam generated in tons to the amount of thermal energy generated in the reaction vessel in MW is in the range of from 1.5 to 7 tₛₜₑₐₘ/MWₕₑₐₜ, preferably in the range of from 1.7 to 5.5 tₛₜₑₐₘ/MWₕₑₐₜ.

In some embodiments of the hydrogenation process, where the heat transfer medium stream HTMS2 is water steam (H₂O_{gaseous}), the ratio of electrical power demand in MWₑₗ to the amount of steam generated in tons is in the range from 0.002 to 0.55 MWₑₗ/tₛₜᵣₑₐₘ, preferred 0.003 to 0.5 MWₑₗ/tₛₜₑₐₘ.

In some embodiments of the hydrogenation process, where the heat pump is an absorption heat pump, preferably an absorption heat pump of category II, the ratio of electrical power demand in MWe to the amount of steam generated in tons is in the range from 0.002 to 0.025 MWₑₗ/tₛₜₑₐₘ: preferred 0.003 to 0.02 MWₑₗ/tₛₜᵣₑₐₘ.

In some embodiments of the hydrogenation process, where the heat pump is a compression heat pump, preferably a compression heat pump to generate steam, the ratio of electrical power demand in MWe to the amount of steam generated in tons is in the range from 0.025 to 0.55 MWₑₗ/tₛₜₑₐₘ, preferred 0.05 to 0.5 MWₑₗ/tₛₜᵣₑₐₘ.

### Compound to be Hydrogenated

The compound to be hydrogenated is preferably a compound having at least one nitro group. In some embodiments of the hydrogenation process, the compound having at least one nitro group (aka the “nitro compound”) is preferably an organic compound having at least one nitro group, preferably selected from the group of nitro alcohol, nitroaromatic and mixtures of nitroalcohol and nitroaromatic.

The nitro compound is preferably an organic compound having at least one nitro group, preferably selected from the group of nitro alcohol, nitroaromatic and mixtures of nitroalcohol and nitroaromatic. In preferred embodiments, the nitro compound for hydrogenation is a nitroaromatic, preferably selected from the group consisting of mononitroaromatic, dinitroaromatic, polynitroaromatic and mixtures of two or more thereof. A “mononitroarimatic” is an aromatic compound having only one nitro group as substituent. A “dinitroaromatic” is an aromatic compound having two nitro groups as substituents. A “polynitroaromatic” in the context of the invention is an aromatic compound having at least three nitro groups. In some preferred embodiments, the compound having at least one nitro group is selected from the group consisting of mononitroaromatic, dinitroaromatic and mixtures of mononitroaromatic and dinitroaromatic, preferably the compound having at least one nitro group comprises at least a dinitroaromatic. A mononitroaromatic is preferably an aromatic compound having in the range of from 6 to 18 carbon atoms and one nitro group as substituent. In some preferred embodiments, the mononitroaromatic is selected from the group consisting of mononitrotoluene, halogen derivatives of mononitrotoluene, mononitrobenzene, the halogen derivatives of mononitrobenzene, mononitroxylene, mononitronaphthalene, nitroaniline and mixtures of two or more thereof. Preferably, the mononitroaromatic is selected from the group consisting of nitrobenzene, o-nitrotoluene, m-nitrotoluene, p-nitrotoluene, 1,2-dimethyl-3-nitrobenzene, 1,2-dimethyl-4-nitrobenzene, 1,4-dimethyl-2-nitrobenzene, 1,3-dimethyl-2-nitrobenzene, 2,4-dimethyl-1-nitrobenzene, 1,3-dimethyl-5-nitrobenzene, 1-nitronaphthalene, 2-nitronaphthalene, o-chloronitrobenzene, m-chloronitrobenzene, p-chloronitrobenzene, 1,2-dichloro-4-nitrobenzene, 1,4-dichloro-2-nitrobenzene, 2,4-dichloro-1-nitrobenzene, 1,2-dichloro-3-nitrobenzene, 4-chloro-2-nitrotoluene, 4-chloro-3-nitrotoluene, 2-chloro-4-nitrotoluene, 2-chloro-6-nitrotoluene, o-nitroaniline, m-nitroaniline, p-nitroaniline and mixtures of two or more thereof. In some preferred embodiments, the mononitroaromatic is selected from the group consisting of mononitrobenzene, halogenated mononitrobenzene, mononitrotoluene, and mixtures of two or more thereof, more preferably selected from the group consisting of nitrobenzene, o-nitrotoluene, m-nitrotoluene, p-nitrotoluene, 1,2-dimethyl-3-nitrobenzene, 1,2-dimethyl-4-nitrobenzene, 1,4-dimethyl-2-nitrobenzene, 1,3-dimethyl-2-nitrobenzene, 2,4-dimethyl-1-nitrobenzene, 1,2-dichloro-4-nitrobenzene, 1,4-dichloro-2-nitrobenzene, 2,4-dichloro-1-nitrobenzene, 1,2-dichloro-3-nitrobenzene and mixtures of two or more thereof, more preferably from the group consisting of o-nitrotoluene, m-nitrotoluene, p-nitrotoluene and mixtures of two or more thereof. A dinitroaromatic is preferably an aromatic compound having in the range of from 6 to 18 carbon atoms. Pref-erably, the dinitroaromatic is selected from the group consisting of dinitrotoluene, halide of dinitrotoluene dinitrobenzene, halide of dinitrobenzene, dinitronaphthalene and mixtures of two or more thereof. In some preferred embodiments, the dinitroaromatic is selected from the group consisting of 1,2-dinitrobenzene, 1,3-dinitrobenzene, 1,4-dinitrobenzene, 2,3-dinitrotoluene, 2,4-dinitrotoluene, 3,4-dinitrotoluene, 3,5-dintritotoluene, 2,6-dinitrotoluene, 3,6-dinitrotoluene, and mixtures of two or more thereof, more preferably from the group consisting of 2,3-dinitrotoluene, 2,4-dinitrotoluene, 3,4-dinitrotoluene, 3,5-dintritotoluene, 2,6-dinitrotoluene, 3,6-dinitrotoluene, and mixtures of two or more thereof.]

In some preferred embodiments of the hydrogenation process, the compound having at least one nitro group comprises 2,4-dinitrotoluene, 2,6-dinitrotoluene or a mixture of 2,4-dinitrotoluene and 2,6-dinitrotoluene. In some preferred embodiments, the compound having at least one nitro group comprises 2,4-dinitrotoluene, 2,6-dinitrotoluene or a mixture of 2,4-dinitrotoluene and 2,6-dinitrotoluene. Industrial mixtures comprising 2,4-dinitrotoluene and 2,6-dinitrotoluene are also suitable, wherein these mixtures preferably comprise at least 55 weight-% 2,4-dinitrotoluene and up to 35 weight-% of 2,6-dinitrotoluene with proportions of preferably up to 5 weight-% of vicinal dinitrotoluene and preferably up to 1.5 weight-% of 2,5- and 3,5-dinitrotoluene based on the overall mixture being 100 weight-%.

The above-mentioned nitro alcohol(s) and nitroaromatic(s) are commercially available. The nitro alcohols and nitroaromatics used may furthermore be obtained by chemical synthesis, such as dinitrotoluenes may be obtained by nitration of toluene for example. The thus formed reaction product usually comprises not only the desired nitro compound but also numerous impurities.

Provided that an above described mixture is employed the weight ratio of an amine compound to water is preferably in the range from 10:1 to 1:10, preferably in the range from 8:1 to 1:5 and particularly preferably in the range from 4:1 to 1:3 and the weight ratio of the amine/water mixture to C1 to C6 monoalcohol is preferably 1000:1 to 1:1, preferably 500:1 to 2.5:1 and particularly preferably 50:1 to 5:1.

The amount of the employed alcoholic solvent, i.e. the C1 to C6 monoalcohol, and of the catalyst-reactivating additions is not restricted in any particular way in the context of the process according to the invention and may be chosen freely as required.

The process according to the invention for hydrogenation of nitro compounds to the corresponding amines may additionally be performed in the absence of solvents. In this procedure the workup of the reaction mixture after the hydrogenation is simplified and side reactions with the solvent are moreover completely inhibited.

### Hydrogen (H₂)

Any gas free of harmful amounts of catalyst poisons such as CO, which contains free hydrogen can be used as hydrogenation gas, for example, a reformer exhaust gas can be used or a mixture of hydrogen with nitrogen and/or carbon monoxid. Preferably, pure hydrogen, i.e. hydrogen having a purity of at least 90%, is used as hydrogenation gas or a mixture of hydrogen and an inert gas, preferably nitrogen, can be used as hydrogenation gas, wherein at least 90 weight-% of the mixture consist of hydrogen and nitrogen, the total weight of the mixture being 100 weight-%.

### Solvent(s)

The conversion of the compound having at least one nitro group into the corresponding compound having at least one amino group is done (within the reactor) in a liquid medium, preferably in a solution or suspension comprising water and optionally one or more C1 to C6 mono alcohol, preferably a C1 to C6 mono alcohol from the group consisting of C1 to C5 mono alcohols, more preferably selected from the group consisting of methanol, ethanol, propanol, including n-propanol and iso-propanol, and mixtures of two or three thereof, more preferably the solvent contains at least iso-propanol. A catalyst-reactivating additive, preferably selected from the group consisting of aprotic solvent, more preferably selected from the group consisting of DMF, dioxane, THF or a mixture of two or more thereof, is used in some embodiments.

### Reaction Conditions

The reactor is operated at a pressure in the range of from 5 to 100 bar, preferably in the range of from 10 to 50 bar, more preferably in the range of from 15 to 40 bar, more preferably in the range of from 20 to 30 bar, and the reaction mixture within the reactor has a temperature in the range of from 50 to 200° C., preferably in the range of from 60 to 180° C., more preferably in the range of from 70 to 150° C.

### Catalyst

The catalyst used in the reaction is preferably a hydrogenation catalyst. Suitable hydrogenation catalysts are known per se for aromatic nitro compounds. It is possible to use homogeneous and/or heterogeneous catalysts, wherein the use of heterogenous catalysts is preferred. The heterogeneous catalysts are employed in the form of particles and are suspended in the reaction suspension. Suitable catalysts are metals from sub-group Vill of the Periodic Table, which are preferably supported on support materials such as activated carbon or oxides of aluminum, silicon or other materials. Preference is given to Raney nickel and/or supported catalysts based on nickel, copper, palladium and/or platinum. Suitable hydrogenation catalysts are known, for example, from WO 00/35852 A1 or WO 2005/037768 A1. The hydrogenation catalyst is preferably used in an amount of 0.01 to 10, preferably 0.1 to 5, particularly preferably 0.2 to 2% by weight, based on the weight of the reaction mixture being 100% by weight.

### 2^{nd} Aspect-Reaction System for Hydrogenation

A second aspect of the invention is directed to a reaction system for hydrogenation of a compound, preferably for converting a compound having at least one nitro group into the corresponding compound having at least one amino group by hydrogenation, comprising:
(a) a reactor having
   (a.1) means for feeding gaseous and liquid materials into the reactor;
   (a.2) means for intermixing in the reactor,
   (a.3) an outlet for removing reaction mixture from the reactor;
   (a.4) at least one inlet for reintroduction of the reaction mixture;
   (a.5) circuit lines outside of the reactor in fluid connection to the outlet (a.3) and inlet (a.4), which enable withdrawal of a reaction mixture from the reactor via outlet (a.3) and reintroduction of the reaction mixture into the reactor via the inlet (a.4);
   (a.6) at least one heat exchanger arranged within the reactor, outside of the reactor or in the circuit line (a.5), wherein the heat exchanger has at least one inlet (a.6.1) for feeding a cooling medium into the heat exchanger and at least one outlet (a.6.2) for removing the cooling medium from the heat exchanger;
(b) a heat pump comprising
   (b.1) at least one inlet (b.1.1) for feeding a cooling medium into the heat pump, and at least one outlet (b.1.2) for removing the cooling medium from the heat pump;
   (b.2) at least one inlet (b.2.1) for feeding a heat transfer medium stream into the heat pump, and at least one outlet (b.2.2) for removing the heat transfer medium stream from the heat pump;

wherein the heat pump is built to allow transfer of thermal energy from a cooling medium stream to a heat transfer medium stream;
(c) lines in fluid connection with the at least one heat exchanger (a.6) and the heat pump (b), which are built and arranged
   (c.1) to allow transfer of the cooling medium stream coming from the at least one outlet of the heat exchanger (a.6.2) into an inlet of the heat pump (b.1.1) and reintroduction of the cooling medium stream coming from the at least one outlet (b.1.2) for removing the cooling medium from the heat pump to the at least one inlet (a.6.1) for feeding a cooling medium into the heat exchanger; and
   (c.2) to allow introduction of a heat transfer medium stream into the at least one inlet (b.2.1) of the heat pump for feeding a heat transfer medium stream into the heat pump and removal of a heat transfer medium stream from the at least one outlet (b.2.2) of the heat pump for removing the heat transfer medium stream of the heat pump.

In some preferred embodiments of the reaction system, the heat pump (b) is an absorption heat pump, preferably an absorption heat pump of category II, or a compression heat pump, more preferably the heat pump of (b) is an absorption heat pump of category II.

In some preferred embodiments of the reaction system, the reactor (a) is selected from the group consisting of (multi) tubular reactor, stirred tank reactor and loop reactor, wherein the reactor is preferably a loop reactor.

All details, embodiments and preferred embodiments disclosed above in the section related to the first aspect apply also for the reaction system, i.e. the second aspect of the invention. The present invention is further illustrated by the following embodiments and combinations of embodiments as indicated by the respective dependencies and back-references. In particular, it is noted that in each instance where a range of embodiments is mentioned, for example in the context of a term such as “The . . . of any of embodiments 1 to 4”, every embodiment in this range is meant to be explicitly disclosed for the skilled person, i.e. the wording of this term is to be understood by the skilled person as being synonymous to “The . . . of any of embodiments 1, 2, 3, and 4”.
1. A hydrogenation process for the hydrogenation of a compound comprising reacting the compound in a liquid medium with hydrogen in the presence of a heterogeneous hydrogenation catalyst in a reaction vessel, the process comprising
   (i) transferring at least a part of the thermal energy generated in the reaction vessel to a cooling medium, preferably a cooling medium stream CMS1, obtaining a cooling medium stream CMS2, which has an increased thermal energy content compared to CMS1;
   (ii) transferring at least a part of the thermal energy comprised in cooling medium stream CMS2 to a heat transfer medium stream HTMS1 in a heat pump HP, thereby obtaining a heat transfer medium stream HTMS2, which has an increased thermal energy content compared to HTMS1.
2. The hydrogenation process of embodiment 1, being a process for the hydrogenation of a compound having at least one nitro group into the corresponding compound having at least one amino group comprising reacting the compound having at least one nitro group in a liquid medium with hydrogen in the presence of a heterogeneous hydrogenation catalyst in a reaction vessel.
3. The hydrogenation process of embodiment 1 or 2, wherein the heat pump of (ii) is an absorption heat pump, preferably an absorption heat pump of category II, or a compression heat pump, more preferably the heat pump of (ii) is an absorption heat pump of category II.
4. The hydrogenation process of any one of embodiments 1 to 3, wherein the heat transfer medium comprises water, preferably at least 90 weight-%, more preferably at least 95 weight-%, more preferably at least 99 weight-% of the heat transfer medium are water, each based on the total weight of the heat transfer medium being 100 weight-%
5. The hydrogenation process of embodiment 4, wherein at least 90 weight-% of heat transfer medium stream HTMS1 are in liquid state and at least at least 90 weight-% of heat transfer medium stream HTMS2 are in gaseous state (H₂O_{gaseous}, water steam).
6. The hydrogenation process of any one of embodiments 1 to 5, wherein heat transfer medium stream HTMS2 has a pressure p₂ in the range of from 0 to 20 barg, preferably in the range of from 0.1 barg to 10 barg.
7. The hydrogenation process of any one of embodiments 1 to 6, wherein the heat transfer medium stream HTMS2 having a pressure p₂ is compressed, preferably by mechanical compression, to give a heat transfer medium stream HTMS2-1 having an increased pressure p₂-1 compared to p₂, wherein p₂-1 is preferably a pressure in the range of from 3 to 40 barg.
8. The hydrogenation process of any one of embodiments 1 to 7, wherein the cooling medium is selected from the group consisting of water, air, solvent and mixtures of two or more thereof, wherein the cooling medium preferably comprises water, preferably at least 90 weight-%, more preferably at least 95 weight-%, more preferably at least 99 weight-% of the heat transfer medium are water, each based on the total weight of the cooling medium being 100 weight-%.
9. The hydrogenation process of any one of embodiments 1 to 8, wherein transferring at least a part of the thermal energy generated in the reaction vessel to cooling medium stream CMS1 in (i) is done by use of a cooling system, which comprises a heat exchanger arranged within the reaction vessel or surrounding the reaction vessel at least partially and a circulating line for introducing CMS1 into the heat exchanger and for removing CMS2 from the heat exchanger.
10. The hydrogenation process of embodiment 9, wherein transferring at least a part of the thermal energy comprised in cooling medium stream CMS2 to a heat transfer medium stream HTMS1 in a heat pump HP in (ii) is done so that a cooling medium stream CMS3 is obtained in (ii), which has the same or higher thermal energy content as CMS1, wherein preferably, CMS3 is reintroduced as CMS1 or at least as part thereof into the cooling system and the heat exchanger respectively.
11. The hydrogenation process of embodiments 9 or 10, wherein the heat exchanger is selected from the group consisting of shell and tube heat exchanger, plate heat exchanger, field tube heat exchanger and coil heat exchanger.
12. The hydrogenation process of any one of embodiments 1 to 11, wherein the reaction vessel is a reactor selected from the group consisting of (multi) tubular reactor, stirred tank reactor and loop reactor, wherein the reaction vessel is preferably a loop reactor.
13. The hydrogenation process of embodiment 12, wherein for the reaction vessel being a loop reactor, the process comprises:
   (i.1) transferring at least a part of the thermal energy generated in the reaction vessel to a cooling medium, preferably a cooling medium stream CMS1, obtaining a cooling medium stream CMS2, which has an increased thermal energy content compared to CMS1;
   (ii.a) feeding CSM2 to a heat exchanger installed in the circulation loop of the loop reactor, thereby transferring a further part of the thermal energy generated in the reaction vessel to CMS2 and obtaining a cooling medium stream CMS4, which has an increased thermal energy content compared to CMS2;
   (ii.b) transferring at least a part of the thermal energy comprised in cooling medium stream CMS4 to a heat transfer medium stream HTMS1 in a heat pump HP, thereby obtaining a heat transfer medium stream HTMS2, which has an increased thermal energy content compared to HTMS1.
14. The hydrogenation process of embodiment 13, wherein the a cooling medium stream CMS3 is obtained in (ii), which has the same or higher thermal energy content as CMS1, wherein preferably, CMS3 is reintroduced as CMS1 or at least as part thereof into the cooling system and the heat exchanger respectively.
15. The hydrogenation process of embodiment 12, wherein for the reaction vessel being a loop reactor, the process comprises:
   (i) transferring at least a part of the thermal energy generated in the reaction vessel to a cooling medium, preferably a cooling medium stream CMS1, obtaining a cooling medium stream CMS2, which has an increased thermal energy content compared to CMS1;
   (ii) sending a part of a cooling medium stream CMS3 (CMS3a) obtained from a heat pump PH to a heat exchanger installed in the circulation loop of the loop reactor, thereby transferring a part of the thermal energy generated in the reaction vessel to said part of cooling medium stream CMS3a and obtaining a partial cooling medium stream CMS5, which has an increased thermal energy content compared to CMS3;
   (iii) combining partial cooling medium stream CMS5 with cooling medium stream CMS2 obtained in (i) thereby obtaining a combined cooling medium stream CMS2+CMS5;
   (iv) feeding the combined cooling medium stream CMS2+CMS5 to the heat pump HP; and transferring at least a part of the thermal energy comprised in combined cooling medium stream CMS2+CMS5 to a heat transfer medium stream HTMS1 in a heat pump HP, thereby obtaining a heat transfer medium stream HTMS2, which has an increased thermal energy content compared to HTMS1.
16. The hydrogenation process of embodiment 15, wherein another part of CMS3 (CMS3b) is reintroduced as CMS1 or at least as part thereof into the cooling system and the heat exchanger respectively.
17. The hydrogenation process of any one of embodiments 1 to 16, wherein the cooling medium stream CMS2 or CMS4 or the combined stream of CSM2 and CSM5, which enters the heat pump HP has a temperature in the range of from 55 to 150° C. and the cooling medium stream CMS3, which leaves the heat pump HP has a temperature in the range of from 30 to 125° C.
18. The hydrogenation process of any one of embodiments 1 to 7, wherein at least 20% of the thermal energy generated in the reaction vessel are transferred, via the cooling medium and the heat pump, to the heat transfer medium, preferably to heat transfer medium stream HTMS1 and heat transfer medium stream HTMS2 respectively.
19. The hydrogenation process of embodiment 18, wherein in case the heat pump being an absorption heat pump, preferably an absorption heat pump of category II, in the range of from 20 to 50% of the thermal energy generated in the reaction vessel are transferred, via the cooling medium and the heat pump, to the heat transfer medium, preferably to heat transfer medium stream HTMS1 and heat transfer medium stream HTMS2 respectively.
20. The hydrogenation process of embodiment 18, wherein in case the heat pump being a compression heat pump, preferably a compression heat pump to generate steam (steam generator), in the range of from 20 to 100%, preferably in the range of from 70 to 100% of the thermal energy generated in the reaction vessel are transferred, via the cooling medium and the heat pump, to the heat transfer medium, preferably to heat transfer medium stream HTMS1 and heat transfer medium stream HTMS2 respectively.
21. The hydrogenation process of any one of embodiments 1 to 20, wherein in case that the heat transfer medium stream HTMS2 is water steam (H₂O_{gaseous}), the ratio of the amount of steam generated in tons to the amount of thermal energy generated in the reaction vessel in MW is in the range of from 0.3 to 7.0 tₛₜₑₐₘ/MWₕₑₐₜ
22. The hydrogenation process of embodiment 21, wherein in case the heat pump being an absorption heat pump, preferably an absorption heat pump of category II, the ratio of the amount of steam generated in tons to the amount of thermal energy generated in the reaction vessel in MW is in the range of from 0.3 to 0.7 tₛₜₑₐₘ/MWₕₑₐₜ, preferably in the range of from 0.35 to 0.6 tₛₜₑₐₘ/MWₕₑₐₜ.
23. The hydrogenation process of embodiment 21, wherein in case the heat pump being a compression heat pump, preferably a compression heat pump to generate steam, the ratio of the amount of steam generated in tons to the amount of thermal energy generated in the reaction vessel in MW is in the range of from 1.5 to 7 tₛₜₑₐₘ/MWₕₑₐₜ, preferably in the range of from 1.7 to 5.5 tₛₜₑₐₘ/MWₕₑₐₜ.
24. The hydrogenation process of any one of embodiments 1 to 23, wherein in case that the heat transfer medium stream HTMS2 is water steam (H₂O_{gaseous}), the ratio of electrical power demand in MWe to the amount of steam generated in tons is in the range from 0.002 to 0.55 MWₑₗ/tₛₜₑₐₘ, preferred 0.003 to 0.5 MWₑₗ/tₛₜₑₐₘ
25. The hydrogenation process of embodiment 21, wherein in case the heat pump being an absorption heat pump, preferably an absorption heat pump of category II, the ratio of electrical power demand in MWe to the amount of steam generated in tons is in the range from 0.002 to 0.025 MWₑₗ/tₛₜₑₐₘ, preferred 0.003 to 0.02 MWₑₗ/tₛₜᵣₑₐₘ.
26. The hydrogenation process of embodiment 21, wherein in case the heat pump being a compression heat pump, preferably a compression heat pump to generate steam, the ratio of electrical power demand in MWe to the amount of steam generated in tons is in the range from 0.025 to 0.55 MWₑₗ/tₛₜₑₐₘ, preferred 0.05 to 0.5 MWₑₗ/tₛₜₑₐₘ.
27. The hydrogenation process of any one of embodiments 1 to 265, wherein the compound having at least one nitro group (aka the “nitro compound”) is preferably an organic compound having at least one nitro group, preferably selected from the group of nitro alcohol, nitroaromatic and mixtures of nitroalcohol and nitroaromatic.
28. The hydrogenation process of embodiment 27, wherein the compound having at least one nitro group comprises 2,4-dinitrotoluene, 2,6-dinitrotoluene or a mixture of 2,4-dinitrotoluene and 2,6-dinitrotoluene.
29. A reaction system for hydrogenation of a compound, preferably for converting a compound having at least one nitro group into the corresponding compound having at least one amino group by hydrogenation comprising:
   (a) a reactor having
      (a.1) means for feeding gaseous and liquid materials into the reactor;
      (a.2) means for intermixing in the reactor,
      (a.3) an outlet for removing reaction mixture from the reactor;
      (a.4) at least one inlet for reintroduction of the reaction mixture;
      (a.5) circuit lines outside of the reactor in fluid connection to the outlet (a.3) and inlet (a.4), which enable withdrawal of a reaction mixture from the reactor via outlet (a.3) and reintroduction of the reaction mixture into the reactor via the inlet (a.4);
      (a.6) at least one heat exchanger arranged within the reactor, outside of the reactor or in the circuit line (a.5), wherein the heat exchanger has at least one inlet (a.6.1) for feeding a cooling medium into the heat exchanger and at least one outlet (a.6.2) for removing the cooling medium from the heat exchanger;
   (b) a heat pump comprising
      (b.1) at least one inlet (b.1.1) for feeding a cooling medium into the heat pump, and at least one outlet (b.1.2) for removing the cooling medium from the heat pump;
      (b.2) at least one inlet (b.2.1) for feeding a heat transfer medium stream into the heat pump, and at least one outlet (b.2.2) for removing the heat transfer medium stream from the heat pump;
   wherein the heat pump is built to allow transfer of thermal energy from a cooling medium stream to a heat transfer medium stream;
   (c) lines in fluid connection with the at least one heat exchanger (a.6) and the heat pump (b), which are built and arranged
      (c.1) to allow transfer of the cooling medium stream coming from the at least one outlet of the heat exchanger (a.6.2) into an inlet of the heat pump (b.1.1) and reintroduction of the cooling medium stream coming from the at least one outlet (b.1.2) for removing the cooling medium from the heat pump to the at least one inlet (a.6.1) for feeding a cooling medium into the heat exchanger; and
      (c.2) to allow introduction of a heat transfer medium stream into the at least one inlet (b.2.1) of the heat pump for feeding a heat transfer medium stream into the heat pump and removal of a heat transfer medium stream from the at least one outlet (b.2.2) of the heat pump for removing the heat transfer medium stream of the heat pump.
30. The reaction system of embodiment 29, wherein the heat pump (b) is an absorption heat pump, preferably an absorption heat pump of category II, or a compression heat pump, more preferably the heat pump of (b) is an absorption heat pump of category II.
31. The reaction system of embodiment 29 or 30, wherein the reactor (a) is selected from the group consisting of (multi) tubular reactor, stirred tank reactor and loop reactor, wherein the reactor is preferably a loop reactor.

The present invention is further illustrated by the following reference examples, comparative examples, and examples.

### EXAMPLES

### Simulations

All simulations were done with the process simulation software ASPEN PLUS™ v.11 or Ebsilon®, in combination with excel-based estimation calculation with specified quality grade, based on FluidExl (LibIF97, IAPWS-IF97) of KCE ThermoFluidProperties. The components used in the process simulation and their characteristics respectively, were taken from the ASPEN PLUS™ v.11 PURE32 Database.

### Definitions, Abbreviations

Pressures indicated in “barg” are related to gauge pressure. Since gauge pressure is measured against the ambient pressure, each pressure value (or range) indicated in barg it is equal to absolute pressure minus atmospheric pressure. “bara” means the absolute pressure in bar.

### Comparative Example 1: Hydrogenation of Dinitro Toluene with Primary and Secondary Cooling Medium Loops

The hydrogenation of dinitro toluene to toluene diamine (mixture comprising about 80 weight-% 2,4-dinitrotoluene and about 20 weight-% of 2,6-dinitrotoluene) was performed in a hydrogenation reactor, which was a loop reactor. To secure a good mixing the reactor content, i.e. the hydrogenation bath, which mainly consisted of water, solvent, solid catalyst and reactands dinitro toluene as well as products toluene diamine was pumped around by an external loop. The reaction heat in the reactor was removed from the hydrogenation bath by a primary cooling medium loop containing a primary cooling medium. The warm primary cooling medium was cooled down back by secondary cooling loop, which was operated with a second cooling medium (for example, river water or air cooler) and the cold primary cooling medium was then sent back to the reactor.

The thermal energy released from the reaction, which had to be taken on by the cooling medium(s), was 40 MW.

### Example 1: Hydrogenation of Dinitro Toluene with Primary Cooling Medium Loop and Heat Pump (Absorption Heat Pump of Category II)

A hydrogenation of dinitro toluene was carried out as in Comparative Example 1. Contrary to Comparative Example 1, no primary cooling medium loop was operated—the setup is shown in FIG. 1. Instead, a heat pump (heat exchanger unit) located in the primary cooling loop was used to recover (at least partially) the reaction heat, i.e. the thermal energy, which was initially transferred to the primary cooling medium, from the primary cooling medium. The reaction heat of the hydrogenation reaction warmed up the primary cooling medium. The warm primary cooling medium stream was then used in the heat pump to generate steam from boiling water. Thereby the primary cooling medium was cooled down and send as cold primary cooling medium steam back to the reactor. A backup cooling system was installed in the primary cooling loop to remove the reaction heat in case of a shutdown of the heat pump and to support during start up or shut down of the reactor system.

An absorption heat pump, so called category II, was used. Thus, the absorption heat was used for generating steam. The desorption energy was used for the cooling of the primary cooling medium. Preferred a LiBr-water mixture was used as heat transfer medium in the absorption heat pump.

The pressure of the generated steam was between 0 barg and 20 barg. About 20 to 50% of the thermal reaction heat of the hydrogenation was transferred into steam. The amount of electrical power was low and the cooling water demand could be reduced by about 30%, compared to the cooling water demand of Comparative Example 1.

The energetic balance according to Example 1 was:

| | |
|---|---|
| tₛₜₑₐₘ/MWₕₑₐₜ: | 0.3-0.7 |
| kWₑₗ/tₛₜₑₐₘ: | 2-25 |
| MW_{cooling}/MWₕₑₐₜ: | 0.4-0.8 |

“tₛₜₑₐₘ” means the amount of steam generated in tons, “MWₕₑₐₜ” means the amount of energy, i.e. the thermal energy generated by the reaction in MW, “kWeₑₗ” means the demand of electricity required for the overall reaction including heat pump in kW, “MW_{cooling}” means the demand of cooling energy in MW.

### Example 2: Hydrogenation of Dinitro Toluene with Primary Cooling Medium Loop and Heat Pump (Compression Heat Pump)

A hydrogenation of dinitro toluene was carried out as in Comparative Example 1. Contrary to Comparative Example 1, no primary cooling medium loop was operated. Instead, a heat pump (heat exchanger unit) located in the primary cooling loop was used to recover (at least partially) the reaction heat, i.e. the thermal energy, which was initially transferred to the primary cooling medium, from the primary cooling medium—the setup is shown in FIG. 1. The reaction heat of the hydrogenation reaction warmed up the primary cooling medium.

The warm primary cooling medium stream was then used in the heat pump to generate steam from boiling water, resulting in heating medium stream HTMS2. Thereby the primary cooling medium was cooled down and send as cold primary cooling medium steam back to the reactor—the temperature of said cooling medium stream CMS1, which went back to the reactor, was also called “Heat waste temperature return”. A backup cooling system was installed in the primary cooling loop to remove the reaction heat in case of a shutdown of the heat pump and to support during start up or shut down of the reactor system.

A compression heat pump, so called category I, was used for steam generation. The evaporation of a solvent/heat transfer medium was used for cooling down the primary cooling medium. After compression of the gaseous solvent/heat transfer medium the condensation of the medium at higher pressures was used for the steam generation.

The experiment was simulated with different heat waste temperature return values of CSM1 and different pressures of the generated steam of HTMS2 between 1 barg and 40 barg. More than 90% of the thermal reaction heat MWₕₑₐₜ of the hydrogenation was transferred into steam. The required amount of electrical power MWe was high, the cooling water demand was low. Outlet temperature from HP back to reactor was between 3° and 100° C.

The energetic balance according to Example 2 was:

| | |
|---|---|
| tₛₜₑₐₘ/MWₕₑₐₜ: | 1.5-7 |
| tₛₜₑₐₘ/(MWₕₑₐₜ + MWₑₗ): | 1-2 |
| MWₑₗ/tₛₜₑₐₘ: | 0.025-0.55 |

### Details of Simulations:

Type of compressor: turbo compressor
Heat pump quality grade (eta=COP_{HP}/COP_{Carnot}): 55%
Overheating of steam: 10 K
Boiler feed temperature: 95° C.

“COP” means the coefficient of performance, calculated based on the equitation COP=Q/W, wherein “Q” is the heat transferred into the heat transfer media (usable heat: HTMS2
HTMS1) and “W” is the work of the compressor (work spent, elelectric energy) or driving heat.

COP_{HP}: Coefficient of performance of the heat pump

COP_{Carnot}: Ideal coefficient of performance calculated based on a Carnot process

The parameters and the resulting values regarding COP_{HP}, MWₑₗ/tₛₜₑₐₘ, tₛₜₑₐₘ/MWₕₑₐₜ and tₛₜₑₐₘ/(MWₑₗ+MWₕₑₐₜ) are shown below in Table 1:

TABLE 1
Parameters and resulting values

| | Heat waste temperature return (CSM1) | | | |
|---|---|---|---|---|
| | 100° C. | | | |
| Steam pressure | MWₑₗ/ | tₛₜₑₐₘ/ | tₛₜₑₐₘ/(MWₑₗ + | |
| (HTMS2) [barg] | tₛₜₑₐₘ | MWₕₑₐₜ | MWₕₑₐₜ) | COP_{HP} |
| 1 | 0.09 | 1.79 | 1.55 | 7.34 |
| 2 | 0.12 | 1.89 | 1.53 | 5.26 |
| 4 | 0.17 | 2.05 | 1.52 | 3.87 |
| 8 | 0.23 | 2.27 | 1.50 | 2.95 |
| 16 | 0.29 | 2.59 | 1.48 | 2.34 |
| 20 | 0.31 | 2.72 | 1.48 | 2.19 |
| 40 | 0.37 | 3.28 | 1.47 | 1.81 |
| | Heat waste temperature return (CSM1) | | | |
| | 90° C. | | | |
| Steam pressure | MWₑₗ/ | tₛₜₑₐₘ/ | tₛₜₑₐₘ/(MWₑₗ + | |
| (HTMS2) [barg] | tₛₜₑₐₘ | MWₕₑₐₜ | MWₕₑₐₜ) | COP_{HP} |
| 1 | 0.12 | 1.89 | 1.55 | 5.52 |
| 2 | 0.15 | 2.00 | 1.53 | 4.28 |
| 4 | 0.20 | 2.17 | 1.52 | 3.33 |
| 8 | 0.25 | 2.41 | 1.50 | 2.64 |
| 16 | 0.31 | 2.77 | 1.48 | 2.15 |
| 20 | 0.33 | 2.92 | 1.48 | 2.03 |
| 40 | 0.40 | 3.55 | 1.47 | 1.71 |
| | Heat waste temperature return (CSM1) | | | |
| | 70° C. | | | |
| Steam pressure | MWₑₗ/ | tₛₜₑₐₘ/ | tₛₜₑₐₘ/(MWₑₗ + | |
| (HTMS2) [barg] | tₛₜₑₐₘ | MWₕₑₐₜ | MWₕₑₐₜ) | COP_{HP} |
| 1 | 0.18 | 2.12 | 1.55 | 3.68 |
| 2 | 0.21 | 2.26 | 1.53 | 3.12 |
| 4 | 0.25 | 2.46 | 1.52 | 2.60 |
| 8 | 0.31 | 2.76 | 1.50 | 2.18 |
| 16 | 0.36 | 3.21 | 1.48 | 1.86 |
| 20 | 0.38 | 3.41 | 1.48 | 1.77 |
| 40 | 0.44 | 4.24 | 1.47 | 1.53 |
| | Heat waste temperature return (CSM1) | | | |
| | 50° C. | | | |
| Steam pressure | MWₑₗ/ | tₛₜₑₐₘ/ | tₛₜₑₐₘ/(MWₑₗ + | |
| (HTMS2) [barg] | tₛₜₑₐₘ | MWₕₑₐₜ | MWₕₑₐₜ) | COP_{HP} |
| 1 | 0.23 | 2.42 | 1.54 | 2.77 |
| 2 | 0.27 | 2.59 | 1.53 | 2.45 |
| 4 | 0.31 | 2.85 | 1.52 | 2.14 |
| 8 | 0.36 | 3.24 | 1.50 | 1.86 |
| 16 | 0.41 | 3.83 | 1.48 | 1.63 |
| 20 | 0.43 | 4.09 | 1.48 | 1.56 |
| 40 | 0.49 | 5.27 | 1.47 | 1.39 |
| | Heat waste temperature return (CSM1) | | | |
| | 30° C. | | | |
| Steam pressure | MWₑₗ/ | tₛₜₑₐₘ/ | tₛₜₑₐₘ/(MWₑₗ + | |
| (HTMS2) [barg] | tₛₜₑₐₘ | MWₕₑₐₜ | MWₕₑₐₜ) | COP_{HP} |
| 1 | 0.29 | 2.82 | 1.55 | 2.21 |
| 2 | 0.32 | 3.04 | 1.53 | 2.02 |
| 4 | 0.36 | 3.38 | 1.52 | 1.81 |
| 8 | 0.41 | 3.91 | 1.50 | 1.62 |
| 16 | 0.46 | 4.75 | 1.48 | 1.45 |
| 20 | 0.48 | 5.13 | 1.48 | 1.40 |
| 40 | 0.54 | 6.96 | 1.47 | 1.27 |

### Example 3: Hydrogenation of Dinitro Toluene with Primary Cooling Medium Loop and Heat Pump (Absorption Heat Pump of Category II) Plus Mechanical Steam Compressor

A hydrogenation of dinitro toluene was carried out as in Example 1, wherein only low pressure steam was generated (pressure 0 barg to 8 barg). In addition to Example 1, compression by an additional mechanical steam compressor was done to pressurize the steam to pressures between 4 barg and 40 barg—the setup is shown in FIG. 2. Additional electrical power input was needed compared to Example 1. The additional power demand compared to Example 1 was MW el, MC/tₛₜₑₐₘ: 0.025-0.3.

### Example 4: Hydrogenation of Dinitro Toluene with Primary Cooling Medium Loop and Heat Pump (Compression Heat Pump) Plus Mechanical Steam Compressor

A hydrogenation of dinitro toluene was carried out as in Example 2, wherein only low pressure steam was generated (pressure 0 barg to 8 barg). In addition to Example 2, compression by an additional mechanical steam compressor was done to pressurize the steam to pressures between 4 barg and 40 barg—the setup is shown in FIG. 2. Additional electrical power input was needed compared to Example 2. The additional power demand compared to Example 2 was MWₑₗ, MC/tₛₜₑₐₘ. 0.025-0.3.

### Details of Simulations:

Type of compressor: turbo compressor
Heat pump quality grade (eta=COP_{HP}/COP Car Carnot): 55%
MC efficiency (isentrope): 75%
Injection pump efficiency: 90%
Overheating of steam: 10 K
Boiler feed temperature: 95° C.

The parameters and the resulting values are shown below in Table 2:

TABLE 2
Parameters and resulting values

| | Heat waste temperature return (CSM1) | | | | | | |
|---|---|---|---|---|---|---|---|
| | 90° C. | | | | | | |
| Steam | Steam | | | | | | |
| pressure | pressure | | | | tₛₜₑₐₘ / | | |
| (HTMS2) | (HTMS2-1) | MW_{el, MC}/ | MW_{el, HP+MC}/ | tₛₜₑₐₘ/ | (MWₑₗ + | | |
| [barg] | [barg] | tₛₜₑₐₘ | tₛₜₑₐₘ | MWₕₑₐₜ | MWₕₑₐₜ) | COP_{HP} | COP_{HP+MC} |
| 0.5 | 4 | 0.08 | 0.17 | 2.02 | 1.51 | 6.92 | 3.93 |
| 0.5 | 16 | 0.17 | 0.24 | 2.29 | 1.47 | 6.92 | 2.77 |
| 0.5 | 40 | 0.23 | 0.29 | 2.55 | 1.46 | 6.92 | 2.3 |
| 1 | 4 | 0.06 | 0.17 | 2.05 | 1.51 | 5.52 | 3.81 |
| 1 | 16 | 0.15 | 0.25 | 2.31 | 1.47 | 5.52 | 2.72 |
| 1 | 40 | 0.21 | 0.30 | 2.57 | 1.46 | 5.52 | 2.28 |
| 4 | 16 | 0.09 | 0.27 | 2.44 | 1.48 | 3.33 | 2.52 |
| 4 | 40 | 0.15 | 0.31 | 2.70 | 1.46 | 3.33 | 2.16 |

### SHORT DESCRIPTION OF THE FIGURES

FIG. 1 shows a setup as used in Example 1 and 2 with a primary cooling medium loop and a heat pump HP (Example 1: absorption heat pump of category II, Example 2: compression heat pump of category I). The hydrogenation of dinitro toluene to toluene diamine is performed in a hydrogenation reactor R. The hydrogenation bath mainly consisting of water, solvents, solid catalyst, reactands and products is pumped around through an external loop 1. For removal of the reaction heat, which is released from the reaction within the reactor R, a primary cooling medium loop comprising loop lines 2, 3 and a heat exchanger located in the reactor (not shown) is used. The warm primary cooling medium stream CMS2 coming from the reactor through loop line 2 comes to the heat pump HP, wherein steam is generated from a heat transfer medium stream HTMS1-here boiling watercoming to the heat pump HP through boiling water feed line 4. The heat transfer medium stream HTMS2, which has an increased thermal energy content compared to HTMS1-here water steam-generated in the heat pump HP leaves through line 5. Due to the heat release in the heat pump HP, the primary cooling medium is cooled down and send as cold cooling medium steam CMS1 back through line 3 to the reactor. A backup cooling system BCS comprising a heat exchanger is installed in the primary cooling loop to remove the reaction heat in case of a shutdown of the heat pump HP and to support during start up or shut down of the reactor system.

FIG. 2 shows a setup as used in Example 3 and 4 with a primary cooling medium loop and a heat pump HP (Example 3: absorption heat pump of category II+mechanical steam compressor, Example 2: compression heat pump of category I+mechanical steam compressor). The hydrogenation of dinitro toluene to toluene diamine is performed in a hydrogenation reactor R. The hydrogenation bath mainly consisting of water, solid catalyst, reactands and products is pumped around through an external loop 1. For removal of the reaction heat, which is released from the reaction within the reactor R, a primary cooling medium loop comprising loop lines 2, 3 and a heat exchanger located in the reactor (not shown) is used. The warm primary cooling medium stream CMS2 coming from the reactor through loop line 2 comes to the heat pump HP, wherein steam is generated from a heat transfer medium stream HTMS1-here boiling water-coming to the heat pump HP through boiling water feed line 4. The heat transfer medium stream HTMS2, which has an increased thermal energy content compared to HTMS1-here water steam-generated in the heat pump HP leaves through line 5. HTMS2 then enters a mechanical steam compressor MSC, wherein the pressure of HTMS2 is increased so that a heat transfer medium stream HTMS2-1, which has an increased pressure compared to HTMS2, leaves MSC via line 6. Due to the heat release in the heat pump HP, the primary cooling medium is cooled down and send as cold cooling medium stream CMS1 back through line 3 to the reactor. A backup cooling system BCS comprising a heat exchanger is installed in the primary cooling loop to remove the reaction heat in case of a shutdown of the heat pump HP and to support during start up or shut down of the reactor system.

FIG. 3 shows a serial setup for increasing the heat pump efficiency by increased temperatures with a primary cooling medium loop and a heat pump HP. The hydrogenation of dinitro toluene to toluene diamine is performed in a hydrogenation reactor R. The hydrogenation bath mainly consisting of water, solvent, solid catalyst, reactands and products is pumped around through an external loop 1. For removal of the reaction heat, which is released from the reaction within the reactor R, a primary cooling medium loop comprising loop lines 2, 2a and 3 and a heat exchanger located in the reactor (not shown) as an additional heat exchanger HE is used. The warm primary cooling medium stream CMS2 coming from the reactor through loop line 2 comes to an additional heat exchanger HE, which is installed in the external loop 1 of the hydrogenation bath. The warm primary cooling medium takes up further heat in HE, whereafter an even warmer primary cooling medium stream CMS4 flows through line 2a in to the heat pump HP. In the heat pump HP, a heat transfer medium stream HTMS2-here steamis generated from a heat transfer medium stream HTMS1-here boiling water-, which comes to the heat pump HP through boiling water feed line 4. The heat transfer medium stream HTMS2, which has an increased thermal energy content compared to HTMS1-here water steam-generated in the heat pump HP leaves through line 5. Due to the heat release in the heat pump HP, the primary cooling medium is cooled down and send as cold cooling medium stream CMS1 back through line 3 to the reactor. A backup cooling system BCS comprising a heat exchanger is installed in the primary cooling loop to remove the reaction heat in case of a shutdown of the heat pump HP and to support during start up or shut down of the reactor system.

FIG. 4 shows a parallel setup for increasing the heat pump efficiency by increased temperatures with a primary cooling medium loop and a heat pump HP. The hydrogenation of dinitro toluene to toluene diamine is performed in a hydrogenation reactor R. The hydrogenation bath mainly consisting of water, solvent, solid catalyst, reactands and products is pumped around through an external loop 1. For removal of the reaction heat, which is released from the reaction within the reactor R, a primary cooling medium loop comprising loop lines 2, 3, 3a and 3b and a heat exchanger located in the reactor (not shown) as an additional heat exchanger HE is used. The cold cooling medium stream CMS1 coming from the heat pump HP is split-off in two streams, wherein a first part is send through line 3 directly back to the reaction, while a second part is sent through line 3a to the additional heat exchanger HE, which is installed in the external loop 1 of the hydrogenation bath. The warm primary cooling medium takes up further heat in HE, whereafter a warmer primary cooling medium stream CMS5 flows through line 3b and is mixed with the warm primary cooling media CMS2. The mixture of CMS2 and CMS5 flows in to the heat pump HP In the heat pump HP, a heat transfer medium stream HTMS2-here steam—is generated from a heat transfer medium stream HTMS1-here boiling water-, which comes to the heat pump HP through boiling water feed line 4. The heat transfer medium stream HTMS2, which has an increased thermal energy content compared to HTMS1-here water steam-generated in the heat pump HP leaves through line 5. Due to the heat release in the heat pump HP, the primary cooling medium stream CMS1 is cooled down and send as cold cooling medium steam back through line 3 to the reactor and 3b to the additional heat exchanger HE. A backup cooling system BCS comprising a heat exchanger is installed in the primary cooling loop to remove the reaction heat in case of a shutdown of the heat pump HP and to support during start up or shut down of the reactor system.

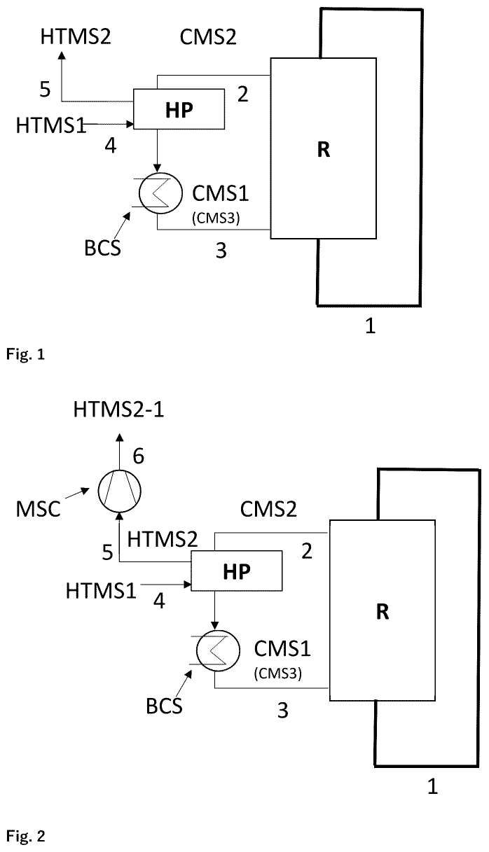

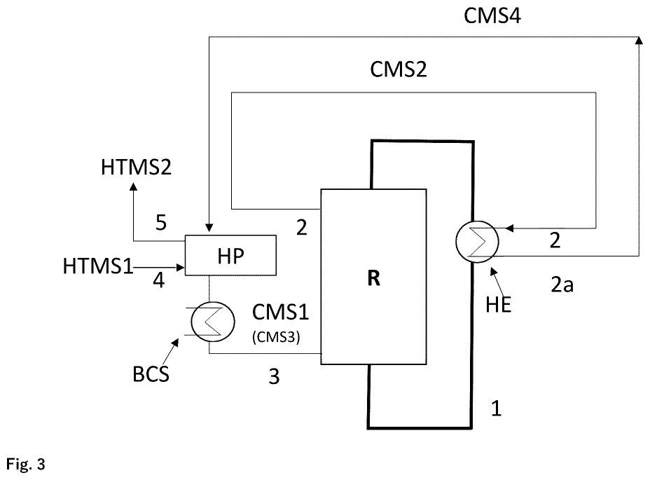

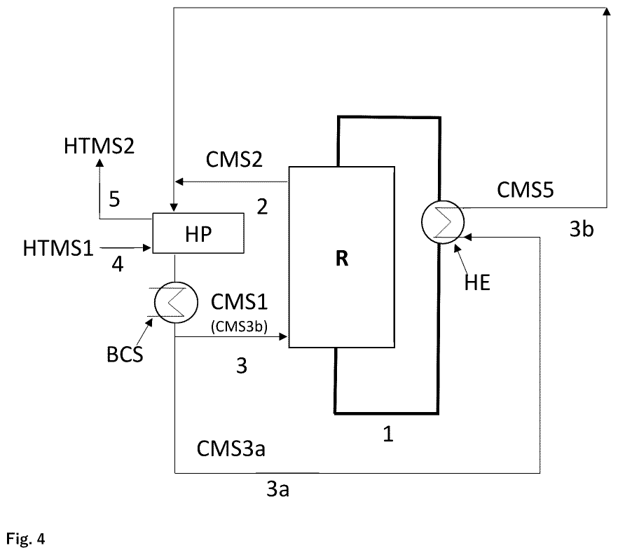

### CITED LITERATURE

WO 00/30743 A1
WO 00/35852 A1
C. Arpagaus et al., Energy 152 (2018), pages 985 to 1010
WO 2014/108352 A1

1-15. (canceled)

16. A process for the hydrogenation of a compound, comprising reacting the compound in a liquid medium with hydrogen in the presence of a heterogeneous hydrogenation catalyst in a reaction vessel, wherein the process further comprises
(i) transferring at least a part of the thermal energy generated in the reaction vessel to a cooling medium, obtaining a cooling medium stream CMS2, which has an increased thermal energy content compared to CMS1; and
(ii) transferring at least a part of the thermal energy comprised in cooling medium stream CMS2 to a heat transfer medium stream HTMS1 in a heat pump HP, thereby obtaining a heat transfer medium stream HTMS2, which has an increased thermal energy content compared to HTMS1.

17. The process of claim 16, being a process for the hydrogenation of a compound having at least one nitro group into the corresponding compound having at least one amino group, comprising reacting the compound having at least one nitro group in a liquid medium with hydrogen in the presence of a heterogeneous hydrogenation catalyst in a reaction vessel.

18. The process of claim 16, wherein the heat pump of (ii) is an absorption heat pump or a compression heat pump.

19. The process of claim 16, wherein the heat transfer medium comprises water.

20. The process of claim 16, wherein heat transfer medium stream HTMS2 has a pressure p₂ in the range of from 0 to 20 barg.

21. The process of claim 16, wherein the heat transfer medium stream HTMS2 having a pressure p₂ is compressed to give a heat transfer medium stream HTMS2-1 having an increased pressure p₂-1 compared to p₂.

22. The process of claim 16, wherein transferring at least a part of the thermal energy generated in the reaction vessel to cooling medium stream CMS1 in (i) is done by use of a cooling system, which comprises a heat exchanger arranged within the reaction vessel or surrounding the reaction vessel at least partially and a circulating line for introducing CMS1 into the heat exchanger and for removing CMS2 from the heat exchanger.

23. The process of claim 16, wherein the reaction vessel is a reactor selected from the group consisting of (multi) tubular reactor, stirred tank reactor and loop reactor.

24. The process of claim 23, wherein for the reaction vessel being a loop reactor, the process comprises:
(i.a) transferring at least a part of the thermal energy generated in the reaction vessel to a cooling medium, obtaining a cooling medium stream CMS2, which has an increased thermal energy content compared to CMS1;
(ii.b) feeding CSM2 to a heat exchanger installed in the circulation loop of the loop reactor, thereby transferring a further part of the thermal energy generated in the reaction vessel to CMS2 and obtaining a cooling medium stream CMS4, which has an increased thermal energy content compared to CMS2; and
(ii.c) transferring at least a part of the thermal energy comprised in cooling medium stream CMS4 to a heat transfer medium stream HTMS1 in a heat pump HP, thereby obtaining a heat transfer medium stream HTMS2, which has an increased thermal energy content compared to HTMS1.

25. The hydrogenation process of claim 23, wherein for the reaction vessel being a loop reactor, the process comprises:
(i) transferring at least a part of the thermal energy generated in the reaction vessel to a cooling medium, obtaining a cooling medium stream CMS2, which has an increased thermal energy content compared to CMS1;
(ii) sending a part of a cooling medium stream CMS3 (CMS3a) obtained from a heat pump PH to a heat exchanger installed in the circulation loop of the loop reactor, thereby transferring a part of the thermal energy generated in the reaction vessel to said part of cooling medium stream CMS3a and obtaining a partial cooling medium stream CMS5, which has an increased thermal energy content compared to CMS3;
(iii) combining partial cooling medium stream CMS5 with cooling medium stream CMS2 obtained in (i) thereby obtaining a combined cooling medium stream CMS2+CMS5; and
(iv) feeding the combined cooling medium stream CMS2+CMS5 to the heat pump HP; and transferring at least a part of the thermal energy comprised in combined cooling medium stream CMS2+CMS5 to a heat transfer medium stream HTMS1 in a heat pump HP, thereby obtaining a heat transfer medium stream HTMS2, which has an increased thermal energy content compared to HTMS1.

26. The hydrogenation process of claim 16, wherein at least 20% of the thermal energy generated in the reaction vessel are transferred, via the cooling medium and the heat pump, to the heat transfer medium.

27. The hydrogenation process of claim 26, wherein for heat pump being an absorption heat pump, in the range of from 20 to 50% of the thermal energy generated in the reaction vessel are transferred, via the cooling medium and the heat pump, to the heat transfer medium.

28. The hydrogenation process of claim 26, wherein for the heat pump being a compression heat pump, in the range of from 20 to 100% of the thermal energy generated in the reaction vessel are transferred, via the cooling medium and the heat pump, to the heat transfer medium.

29. A reaction system for hydrogenation of a compound, comprising:
(a) a reactor having
(a.1) means for feeding gaseous and liquid materials into the reactor;
(a.2) means for intermixing in the reactor,
(a.3) an outlet for removing reaction mixture from the reactor;
(a.4) at least one inlet for reintroduction of the reaction mixture;
(a.5) circuit lines outside of the reactor in fluid connection to the outlet (a.3) and inlet (a.4), which enable withdrawal of a reaction mixture from the reactor via outlet (a.3) and reintroduction of the reaction mixture into the reactor via the inlet (a.4);
(a.6) at least one heat exchanger arranged within the reactor, outside of the reactor or in the circuit line (a.5), wherein the heat exchanger has at least one inlet (a.6.1) for feeding a cooling medium into the heat exchanger and at least one outlet (a.6.2) for removing the cooling medium from the heat exchanger;
(b) a heat pump comprising
(b.1) at least one inlet (b.1.1) for feeding a cooling medium into the heat pump, and at least one outlet (b.1.2) for removing the cooling medium from the heat pump;
(b.2) at least one inlet (b.2.1) for feeding a heat transfer medium stream into the heat pump, and at least one outlet (b.2.2) for removing the heat transfer medium stream from the heat pump;
wherein the heat pump is built to allow transfer of thermal energy from a cooling medium stream to a heat transfer medium stream;
(c) lines in fluid connection with the at least one heat exchanger (a.6) and the heat pump (b), which are built and arranged
(c.1) to allow transfer of the cooling medium stream coming from the at least one outlet of the heat exchanger (a.6.2) into an inlet of the heat pump (b.1.1) and reintroduction of the cooling medium stream coming from the at least one outlet (b.1.2) for removing the cooling medium from the heat pump to the at least one inlet (a.6.1) for feeding a cooling medium into the heat exchanger; and
(c.2) to allow introduction of a heat transfer medium stream into the at least one inlet (b.2.1) of the heat pump for feeding a heat transfer medium stream into the heat pump and removal of a heat transfer medium stream from the at least one outlet (b.2.2) of the heat pump for removing the heat transfer medium stream of the heat pump.

30. The reaction system of claim 29, wherein the reaction system converts a compound having at least one nitro group into the corresponding compound having at least one amino group by hydrogenation.

31. The reaction system of claim 29, wherein the heat pump (b) is an absorption heat pump.
